(19) United States
(12) Patent Application Publication
(10) Pub. No.: US 2011/0195514 A1
(43) Pub. Date: Aug. 11, 2011

(54) COLORIMETRIC REAGENT FOR PREVENTION OF PEROXIDE FORMATION IN SOLVENTS

(76) Inventors: ALLEN WALLACE APBLETT, Stillwater, OK (US); NICHOLAS FERDINAND MATERER, Stillwater, OK (US)

(21) Appl. No.: 13/021,200

(22) Filed: Feb. 4, 2011

Related U.S. Application Data
(60) Provisional application No. 61/301,686, filed on Feb. 5, 2010.

(51) Int. Cl. G01N 33/00

(52) U.S. Cl. 436/135

(57) ABSTRACT

A reagent comprising a reduced metal oxide in a carrier for exposure to a solvent for the detection of the presence of peroxide in the solvent is disclosed. The reduced metal oxide may be molybdenum bronze. The reagent may have a dark color before exposure to peroxide and have a light color after exposure to peroxide.

### CROSS REFERENCE TO RELATED APPLICATIONS

This application claims the priority of U.S. Provisional Patent Application No. 61/301,686 entitled “COLORIMETRIC REAGENT FOR PREVENTION OF PEROXIDE FORMATION IN SOLVENTS,” filed Feb. 5, 2010, the contents of which are hereby incorporated by reference.

### STATEMENT REGARDING FEDERALLY SPONSORED RESEARCH OR DEVELOPMENT

This invention was made with government support under grant ECCS-0731208 awarded by The National Science Foundation. The government has certain rights in the invention.

### FIELD OF THE INVENTION

This disclosure relates to explosives detection in general and, more specifically, to detection of peroxide explosive in solvents.

### BACKGROUND OF THE INVENTION

A peroxide is an organic compound that contains one or more peroxide functional groups (R—O—O—R or ROOH). Peroxides can display an explosive power that is on a par with high explosives and they are usually very unstable. They are highly sensitive to heat, friction, shock, and impact. Thus, the formation of these compounds in solvents creates an extreme hazard.

Numerous solvents become dangerously explosive due to reaction with oxygen to form highly explosive peroxides. These must be routinely tested or discarded. Often organic compounds are added to solvents to prevent peroxide formation; but these can strongly interfere with chemical reactions and analyses. As a result, they are used in small quantities that do not extend the solvent's recommended shelf life beyond a few months.

What is needed is a system and method for addressing the above and related issues.

### SUMMARY OF THE INVENTION

The invention of the present disclosure, in one aspect thereof, comprises a method of detecting solvent peroxidation. The method includes exposing the solvent to a peroxide removing agent, the agent providing a non-explosive hydrogen reducing capability accompanied by a color change when the reaction occurs, and inspecting the reagent to determine whether a color change has occurred.

In some embodiments, exposing the solvent to a peroxide removing agent comprises passing the solvent through a column containing the peroxide removing agent. In other embodiments this comprises placing the agent on a catalyst support pellet that is placed in the solvent. In some embodiments, the agent changes from dark blue to pale yellow in the presence of peroxide. The agent may reduce peroxides into an alcohol group and water. The agent may comprises a molybdenum bronze.

The invention of the present disclosure, in another aspect thereof, comprises a reagent providing a reduced metal oxide in a carrier for exposure to a solvent for the detection of the presence of peroxide in the solvent. The reduced metal oxide may be molybdenum bronze. The reagent may have a dark color before exposure to peroxide and have a light color after exposure to peroxide.

### BRIEF DESCRIPTION OF THE DRAWINGS

FIG. 1: Formation of peroxides by diethyl ether.

### DETAILED DESCRIPTION OF THE PREFERRED EMBODIMENTS

In one embodiment, the product of the present disclosure is a non-contaminating dark blue reagent that can be added to solvents to remove peroxides. Unlike other technologies for preventing peroxide formation, the reagent of the current disclosure provides a visual indication that the solvent is safe to use. Once the reagent becomes exhausted, a color change from dark blue to pale yellow will occur. Thus, the presence of blue reagent will demonstrate the absence of peroxides. In another embodiment, additional reagent may be added over time to ensure that there is no accumulation of explosives.

The product will have another major advantage in that it will be not contaminate solvents with organic compounds and their oxidation products. Thus, the product is a boon to analytical laboratories that struggle with such contaminants when performing trace analyses for pesticides, drugs, metabolites, etc. Furthermore solvents will not need to be purified to remove organic antioxidants in cases where these compounds interfere with the desired reactions.

The slow reactions of organic substances with oxygen in the air leading to undesirable products are familiar to all of us. The phenomena may be observed in such various occurrence's as the spoiling of fruit and food, oils becoming rancid, and even the aging of human beings. A significant number of laboratory solvents can undergo these autoxidation reactions under normal storage conditions to form unstable and potentially dangerous peroxide by-products [2-4]. These reactions occur when susceptible materials are exposed to atmospheric oxygen and are catalyzed by heat and light.

The molecular structure of the solvent is the primary factor that controls its propensity for hazardous peroxide formation. Potential peroxide-forming solvents are typically classified into two categories on the basis of their susceptibility for peroxide formation. Each of these categories is associated with general handling and use guidelines [2]. The two categories relevant to solvents are (1) Class A solvents, that pose a peroxide related safety risk without having to be pre-concentrated and (2) Class B solvents, those that require concentration of the solvent by evaporation or distillations before the peroxides begin to pose a safety hazard. (Note, however, that the solvents may be in a condition that is unacceptable for DNA, protein, or trace chemical analysis even if an immediate explosion hazard is not present.) Exemplary storage time guidelines for previously opened solvent containers are provided in Table 1. However, these storage times are based on ideal conditions in which the solvents are continually stored in opaque containers and under inert atmospheric gases.

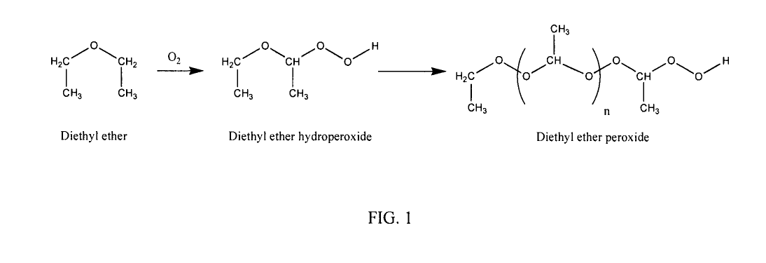

The solvents that are prone to peroxide formation include many that are commercially and industrially very important. For example diethyl ether is a valuable solvent used extensively for organic extractions and the synthesis of compounds and pharmaceuticals. Diethyl ether is particularly useful and important for a type of reagent called a Grignard reagent, which is widely used by academia and industry for alkylation reactions. When exposed to air and light it first forms a hydroperoxide (FIG. 1) that subsequently converts via polymerization to diethyl ether peroxide. Diethyl ether peroxide is a colorless oily liquid that is an extremely powerful and friction sensitive explosive such that an amount of less than 5 milligrams can damage a chemical apparatus.

A peroxide is an organic compound that contains one or more peroxide functional groups (R—O—O—R or ROOH). Peroxides can display an explosive power that is on a par with high explosives and they are usually very unstable. They are highly sensitive to heat, friction, shock, and impact. Thus, the formation of these compounds in solvents creates an extreme hazard.

TABLE 1
Partial List of Peroxide-Forming Solvents [2]
Class A: Peroxide Hazard From Storage

| | | |
|---|---|---|
| Butadiene | Chloroprene | Divinyl acetylene |
| Isopropyl ether | Vinylidene chloride | Tetrafluoroethylene |

Chemicals should be tested for peroxide formation before using
or discarded after 3 months.

Class B Peroxide Hazard from Concentration

| | | |
|---|---|---|
| Acetalaldehyde | Benzyl alcohol | Chlorofluoroethylene |
| Cumene | Cyclohexene | 2-Cyclohexen-1-ol |
| Cyclopentene | Decahydronaphthalene | Diacetylene |
| Dicyclopentadiene | Diethylene glycol | |
| | dimethyl ether | |
| Dioxane | Ethyl ether | Furan |
| 4-Heptanol | 2-Hexanol | Methyl acetylene |
| 3-Methyl-1-butanol | Methyl-isobutyl ketone | Methylcyclopentane |
| 2-Pentanol | 4-Penten-1-ol | Phenylethanol |
| Tetrahydrofuran | Tetrahydronaphthalene | Vinyl ethers |
| Other secondary alcohols | | |

Chemicals should be tested for peroxides before distillation or evaporation
and tested for peroxide formation or discarded after 1 year.

The most used method of solvent protection is the addition of an inhibitor to prevent peroxide formation. For the majority of peroxide-forming solvents, butylated hydroxy toluene (BHT) is commonly used for this purpose. BHT ‘scavenges’ free radicals generated by autoxidation reactions and derails the formation of peroxides. However, over time, the BHT or other inhibitor in the solvent can become exhausted, thus allowing peroxides to form. Also, distilling the solvent can completely remove the BHT and make the solvent immediately susceptible to peroxide formation.

Unfortunately, free radical scavengers such as BHT can only be used whenever the presence of the stabilizing species does not interfere with intended application. There are many instances where low levels of free radical scavengers interfere with the intended application of the solvent. This can include several types of chemical reactions, polymerization reactions, and trace analysis. In these cases, the solvent must be purified prior to use and utilized quickly. In one embodiment, the reagent of the present disclosure will tremendously reduce the risk and added costs of such applications.

Another method for removing peroxides from solvents is passing them through a column of alumina. While this produces peroxide-free solvents for immediate use, it also concentrates the peroxides on an alumina column, creating a severe explosion hazard that must be neutralized quickly by a chemical treatment before it dries out. In one embodiment, the reagent product of the present disclosure may be employed in the same manner as the alumina column, but all of the peroxide explosion hazard would be eliminated. The color change that occurs as the presently disclosed reagent reacts with peroxides will ensure that the remaining capacity of column will be visually observable, thus preventing any possibility of escape of peroxides in the effluent from an overloaded-column.

According to the present disclosure, nanometric inks may be utilized for identification and sensing of hydrogen peroxide and peroxide-based explosives. These inks are based on molybdenum hydrogen bronze. In one embodiment, this is produced by reduction of molybdenum trioxide with hydrogen or a hydrogen source such as an alcohol [5-10]. The nanometric inks represent a highly colored compound that contains pentavalent metal centers with attached hydroxides. The latter species have the unusual property of reacting as if they are hydridic rather than protic like a normal hydroxide [6]. This is a result of the fact that transfer of the hydrogen ion to a substrate is usually accompanied by electron transfer and reoxidation of the metal ion to the hexavalent state.

In some respects, the bronze can be considered to be a convenient non-explosive storage medium for reactive hydrogen [11,12]. Thus, this reagent can act as a hydrogen transfer or reducing agent when contacted by the strongly oxidizing species (such as nitrogen oxides, chlorate, and peroxides). Such oxidizing species are used in, for example, improvised and conventional explosives. Notably, the reactions with the reagent of the present disclosure are accompanied by a marked color change from dark blue to pale yellow. This provides a colorimetric identification of explosives. A complete color change away from blue toward pale yellow is indicative that the reagent has been exhausted and is no longer functioning to remove and contain reactive hydrogen.

In removal of peroxides from solvents, the reagent of the present disclosure works by reducing the peroxides present in the solvent to alcohol groups and water (Equation 1).

2HMo₂O₆+ROOH→2MoO₃+ROH+H₂O (Eq. 1)

For the purpose of solvent protection, the molybdenum bronzes can be used directly in a powder, bead, wire, or pellet form. In another embodiment, they can be dispersed in water and then used to coat an inert support such as catalyst support pellets, calcium sulfate pellets, etc.

The alkali metal molybdenum bronzes can be derived from the hydrogen bronze by reaction with alkali, or can be prepared directly via a variety of other methods. A lithium derivative is particularly suitable due to easy suspension in water to give a concentrated solution. This can be prepared by stirring either a sodium molybdenum bronze with an aqueous lithium salt (e.g. lithium chloride) or a molybdenum hydrogen bronze with lithium hydroxide or lithium carbonate. In another embodiment, support pellets coated with molybdenum oxide can be prepared by impregnation with a solution of a molybdate or polymolybdate salt (ammonium salts work well) followed by heating to convert the salts to MoO₃. The material thus prepared is then treated with a reducing agent to convert the molybdenum trioxide to a hydrogen or sodium bronze.

In addition to molybdenum bronzes, other reduced metal oxides can be used for the purpose of preventing peroxide formation in solvents. These includes the oxides of the Group VB and VIB transition metals either as bronzes or stoichiometric lower oxides that both react with peroxide and dramatically change color as this reaction takes place. Examples include, but are not limited to, VO₂, MₓWO₃, MₓV₂O₅, MₓMoO₃, MoO₂, MₓTa₂O₅, (M=alkali metal or H) and others.

Various embodiments of the reagents disclosed herein may be useful to university chemistry and biochemistry laboratories, pharmaceutical manufacturers and research laboratories, chemical manufactures, and analytical laboratories, and other enterprises. Notably the chemical industry avoids the use of diethyl ether and other peroxide forming ethers like tetrahydrofuran (THF) or ethylene glycol dimethyl ether (1,2-dimethoxyethane) although they are superior solvents for many processes. Removing the danger of ether peroxides may allow industry to realize cost savings through the use of solvents better suited to their processes.

Currently, the pharmaceutical industry has placed emphasis on recycling and reuse of solvents, a procedure that could be economically and safely applied to peroxide-forming solvents through the use of reagents of the present disclosure. It has also been demonstrated that peroxides in solvents are very problematic for trace protein sequencing and DNA analysis since the peroxides degrade the biomolecules [1]. Thus, the use of guaranteed peroxide-free solvents using the various embodiments of the reagents disclosed herein would enhance the ability to perform such analyses dependably. The absence of organic antioxidants and their by-products would also be extremely beneficial for the performance of trace analysis for pesticides, metabolites, etc.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

### REFERENCES

1. Brown, K. “Peroxides in Protein Sequencing Solvents: Effects of PTH-Amino Acids and How to Detect Them” ABRF News Metholology Articles, 1995, 6 (2) 13-15.
2. Jackson, H. L., McCormack, W. B., Rondestvedt, C. S., Smeltz, K. C., and Viele, I. E. “Control of Peroxidizable Compounds”, J. Chem. Educ., 1970, 46 (3), A175.
3. Kelly, R. J, Review of Safety Guidelines for Peroxidizable Organic Compounds, Chemical Health and Safety, 1996, 3 (5), 28-36.
4. Clark, D. E., Peroxides and Peroxide—Forming Compounds, Chemical Health and Safety, 2001, 8 (5), 12-21.
5. Sotani, N.; Eda, K.; Kunitomo, M., Hydrogen insertion compounds omolybdenum trioxide (hydrogen molybdenum bronze, HxMoO3). Trends in Inorganic Chemistry 1990, 1, (1), 23-39.
6. Fripiat, J. J., Hydrogen bronzes: a review of some of their physical and catalytic properties. NATO ASI Series, Series C: Mathematical and Physical Sciences 1983, 105, (Surf. Prop. Catal. Non-Met.), 477-91.
7. Tinet, D.; Fripiat, J. J., Hydrogen bronzes formation process, structure and physical properties. Solid State Protonic Conduct. 1 Fuel Cells Sens., Dan.-Fr. Workshop \“Solid State Mater. Low Medium Temp. Fuel Cells Monit., Spec. Emphasis Proton Conduct.\” 1982, 259-68.
8. Klavins, J.; Millere, I., Hydrogen tungsten oxide bronzes. Latvijas PSR Zinatnu Akademijas Pestis, Kimijas Serija 1980, (4), 387-401.
9. Schwarzmann, E.; Birkenberg, R., Hydrogen analogs of tungsten bronzes. Zeitschrift fuer Naturforschung, Teil B: Anorganische Chemie, Organische Chemie, Biochemie, Biophysik, Biologie 1971, 26, (10), 1069-70.
10. Kihlborg, L., Tungsten bronzes and related compounds. Studies in Inorganic Chemistry 1983, 3, (Solid State Chem.), 143-52.
11. Apblett, A. W.; Kiran, B. P.; Oden, K., Reductive Dechlorination of Chloromethanes Using Tungsten and Molybdenum Hydrogen Bronzes or Sodium Flypophosphite. In Chlorinated Solvents and DNAPLS; Reactive Permeable Barriers and Other Innovations, ACS Book Series: Washington, 2001; pp 154-164.
12. Bollapragada, P. K. S. Chemical transformations using tungsten and molybdenum hydrogen bronzes. Ph.D. Thesis, Oklahoma State University, Stillwater, 2003.

What is claimed is:

1. A method of detecting solvent peroxidation, comprising:
exposing the solvent to a peroxide removing agent, the agent providing a non explosive hydrogen reducing capability accompanied by a color change when the reaction occurs; and
inspecting the reagent to determine whether a color change has occurred.

2. The method of claim 1, wherein exposing the solvent to a peroxide removing agent comprises passing the solvent through a column containing the peroxide removing agent.

3. The method of claim 1, wherein exposing the solvent to a peroxide removing agent comprises placing the agent on a catalyst support pellet that is placed in the solvent.

4. The method of claim 1, wherein the agent changes from dark blue to pale yellow in the presence of peroxide.

5. The method of claim 1, wherein the agent reduces peroxides into an alcohol group and water.

6. The method of claim 1, wherein the agent comprises a molybdenum bronze.

7. A method of preparing a peroxide detecting and removal agent comprising reducing a metal oxide to create an agent that non explosively reacts with peroxides to form an alcohol group and water and is accompanied by a color change during the reaction with the peroxide.

8. The method of claim 8, further comprising preparing a column of the agent and passing a solvent therethrough, and observing the column for the color change.

9. The method of claim 7, further comprising dispersing the agent in water and coating an inert support with the dispersed agent.

10. The method of claim 7, wherein the reduced metal oxide is group VB metal.

11. The method of claim 7, wherein the reduced metal oxide group is a VIB metal.

12. The method of claim 7, wherein the reduced metal oxide group is a bronze.

13. The method of claim 7, wherein the reduced metal oxide group is stoichiometrically lower than a bronze.

14. The method of claim 7, wherein the reduced metal oxide comprises molybdenum bronze.

15. A reagent comprising a reduced metal oxide in a carrier for exposure to a solvent for the detection of the presence of peroxide in the solvent.

16. The reagent of claim 15, wherein the reduced metal oxide is molybdenum bronze.

17. The reagent of claim 15, wherein the reagent has a dark color before exposure to peroxide and has a light color after exposure to peroxide.
